(19) United States
(12) Patent Application Publication
(10) Pub. No.: US 2007/0122351 A1
(43) Pub. Date: May 31, 2007

(54) Inhalation Drug Combinations

(75) Inventors: Robert Leonard KUNKA, Research Triangle Park, NC (US); Tushar Pannalal SHAH, Research Triangle Park, NC (US)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(73) Assignee: GLAXO GROUP LIMITED, Greenford (GB)

(21) Appl. No.: 11/668,647

(22) Filed: Jan. 30, 2007

Related U.S. Application Data
(62) Division of application No. 10/472,407, filed on Mar. 5, 2004.
(60) Provisional application No. 60/277,229, filed on Mar. 20, 2001.

(51) Int. Cl. A61K 9/12; A61K 31/573

(52) U.S. Cl. 424/45; 514/171

(57) ABSTRACT

A method for treating respiratory disorders by administrating by inhalation an effective amount of a β₂-receptor agonist, an acceptable amount of a corticosteriod, and HFA 134a, to a patient in need thereof, is disclosed. Preferably, the β₂-receptor agonist is salmeterol or a physiologically acceptable salt thereof, and the corticosteriod is fluticasone propionate or a solvate thereof. The combination of salmeterol, fluticasone proprionate, and HFA 134a may lower the risk of cardiac arrhythmias, sudden death, or hypercorticism that are sometimes associated with the simultaneous administration of a β₂-receptor agonist and an anti-inflammatory corticosteroid.

### FIELD OF THE INVENTION

The present invention relates to treatment of patients with inhaled drug combinations.

### BACKGROUND

Asthma is a condition characterized by variable, reversible obstruction of the airways, which is caused by a complex inflammatory process within the lungs. The administration of a long acting β₂-receptor agonist by inhalation has been used successfully as a treatment for asthma. The β₂-receptor agonist works by dilating the bronchial airways. It has also long been recognized that the administration of a prophylactic anti-inflammatory corticosteroid is useful to minimize inflammation of the bronchial pathways. Long acting β₂-receptor agonists and corticosteroids therefore have complementary modes of action of airway smooth muscle and inflammation, respectively. Thus, the co-administration of a corticosteroid and a long acting β₂-receptor agonist, particularly fluticasone propionate and salmeterol, is an effective treatment for asthma and other respiratory disorders.

Both salmeterol and fluticasone propionate are well-established products in many countries. The administration of salmeterol and fluticasone propionate simultaneously, sequentially, or separately by inhalation using a metered dose inhaler (MDI) has been described in U.S. Pat. No. 5,270,305, the entire contents of which are hereby incorporated by reference. Currently, salmeterol and fluticasone propionate are available commercially as individual MDI products containing CFC propellant P11/12. The recommended therapeutic dose of salmeterol by MDI is 42 μg bid (dose expressed as ex-actuator). For fluticasone propionate, the recommended therapeutic doses in adults range from 88 μg to 880 μg bid depending on the severity of the patient's asthma.

Treatment with a corticosteroid and a long acting β₂-receptor agonist by inhalation may provide optimal therapy for asthma in patients who require therapy with both classes of drugs. To aid compliance in patients who need regular treatment with both types of drugs and to provide improved control of asthma for patients who are not stable on the administration of only one type of drug, a combination product of salmeterol xinafoate, a long acting β₂-antagonist, and fluticasone propionate, a potent topical corticosteroid, was developed (see, for example, U.S. Pat. No. 5,270,305). This product is being marketed as SERETIDE® Diskus (in which the drugs are administered in a powder form), and SERETIDE®-HFA (in which the drugs are administered from a metered dose inhaler (MDI) which uses HFA-134a as a propellant).

Salmeterol xinafoate (4-hydroxy-α¹-(((6-(4-phenylbutoxy)hexyl)amino)methyl)-1,3-benzenedimethanol, 1-hydroxy-2-naphthalenecarboxylate) is a bronchodilator having an extended duration of activity and is described in U.S. Pat. No. 5,676,929 (the entire contents of which is hereby incorporated by reference). Fluticasone propionate (S-(fluoromethyl)6α,9-difluro-11β,17-dihydroxy-16α-methyl-3-oxoandrosta-1,4-diene-17β-carbothioate,17-propionate) is a topical anti-inflammatory corticosteroid also described in U.S. Pat. No. 5,676,929.

Although there are no data available to date on the effects of acute or chronic overdose with inhaled fluticasone propionate, it is known within the art that the use of corticosteroids may produce serious side effects. Such signs or symptoms are generally dose dependent and may include musculoskeletal effects (including osteoporosis, myopathy, aseptic necrosis of bone), opthalmic effects (including posterior subcapsular cataracts), gastrointestinal effects (including ulcers, pancreatitis, nausea, vomiting), cardiovascular effects (hypertension, atherosclerosis), central nervous system effects (pseudotumor cerebri, psychiatric reactions), dermatological effects (hirsutism, redistribution of subcutaneous fat, impaired wound healing, thinning of the skin) and suppression of the hypothalamus-pituitary-adrenal axis. Further, it is known in the art that chronic overdose of fluticasone propionate may result in hypercorticism.

Overdose of salmeterol may be expected to result in exaggeration of the pharmacologic adverse effects associated with β₂-receptor agonists, including tachycardia and/or arrhythmia, tremor, headache, and muscle cramps. Overdose of salmeterol can lead to clinically significant prolongation of the QTc interval, which can produce ventricular arrhythmias. Other signs of overdose may include hypokalemia and hyperglycemia. Although these side effects are rare at standard therapeutic dosages, the potential still exists for some patients to experience adverse effects from these medications.

### SUMMARY OF THE INVENTION

Surprisingly, the present inventors have found that simultaneous administration of salmeterol and fluticasone propionate by inhalation with the propellant HFA 134a, lowers negative systemic side effects usually associated with administration of either drug, as well as increases the efficacy of the drugs. Specifically, the co-administration of salmeterol and fluticasone propionate by a HFA propellant resulted in lower fluticasone propionate and salmeterol systemic exposure, which in turn led to reduced urinary lower cortisol excretion and a reduction in the increase in heart rate and QTc interval, when compared to inhalation of either drug alone by a CFC-based inhaler. Thusly, the co-administration of salmeterol and fluticasone propionate by a HFA propellant may reduce the risk of HPA axis effects and cardiac arrhythmias in asthmatic patients, in addition to providing instant relief from spasm and inflammation of the bronchial pathways.

The level of either drug in the bloodstream has been found to be decreased when compared to either product administered alone with a CFC propellant. Thus, the present invention provides a method for treating asthma and other respiratory disorders with an opportunity to reduce the negative side effects usually associated with the separate administration of salmeterol and fluticasone propionate.

Therefore, in one embodiment, the present invention is directed to a method for decreasing the systemic exposure of a drug combination comprising at least two drugs in a patient comprising the step of administering by inhalation to a patient in need thereof a pharmaceutical composition comprising an effective amount of at least two drugs in a HFA propellant.

In another embodiment, the present invention is directed to a method for decreasing side effects of a drug combination comprising at least two drugs in a patient comprising the step of administering by inhalation to a patient in need thereof an effective amount of a pharmaceutical composition comprising at least two drugs and a HFA propellant.

In another embodiment, the present invention is directed to a method for reducing hypercorticism in a patient, particularly a patient that is sensitive to hypercorticism, comprising the step of administrating by inhalation to a patient in need thereof a pharmaceutical composition comprising an effective amount of a β₂-receptor agonist, such as salmeterol or a physiologically acceptable salt thereof, an effective amount of a corticosteroid, such as fluticasone propionate or a solvate thereof, and HFA 134a.

In another embodiment, the present invention is directed to a method for reducing the potential increase in heart rate in a patient, particularly an asthma patient that has been diagnosed as having an increased heart rate, comprising the step of administrating by inhalation to a patient in need thereof a pharmaceutical composition comprising an effective amount of a β₂-receptor agonist, such as salmeterol or a physiologically acceptable salt thereof, an effective amount of a corticosteroid, such as fluticasone propionate or a solvate thereof, and HFA 134a.

In another embodiment, the present invention is directed to a method for potentially reducing the risk of cardiac arrhythmia or sudden death in a patient, particularly an asthma patient sensitive to β₂-receptor agonists, comprising an effective amount of a β₂-receptor agonist, such as salmeterol or a physiologically acceptable salt thereof, an effective amount of a corticosteroid, such as fluticasone propionate or a solvate thereof, and HFA 134a.

In another embodiment, the present invention is directed to a method of prescribing medication to an asthma patient comprising:

a) investigating the patient's susceptibility to or history of increased heart rate and/or cardiac arrhythmia; and

b) prescribing to said patient a pharmaceutical inhalation formulation comprising an effective amount of a β₂-receptor agonist, such as salmeterol or a physiologically acceptable salt thereof, an effective amount of a corticosteroid, such as fluticasone propionate or a solvate thereof, and HFA 134a, based in part on the objective of minimizing problems associated with increased heart rate, and/or cardiac arrhythmia.

This method may also include the further step of:

c) administering the pharmaceutical formulation to the patient according to the prescription of step b).

In another embodiment, the present invention is directed to a packaged inhaler for treating asthma, comprising an aerosol drug dispensing device; a pharmaceutical formulation comprising an effective amount of a β₂-receptor agonist, such as salmeterol or a physiologically acceptable salt thereof, an effective amount of a corticosteroid, such as fluticasone propionate or a solvate thereof, and HFA 134a contained in said aerosol drug dispensing device; and printed information associated with said drug dispensing device which describes at least one of the following: less systemic exposure to said drug product and decreased side effects of said drug formulation.

In another embodiment, the present invention is directed to a method for promoting a pharmaceutical composition for treating asthma comprising: distributing information to the public or to doctors which indicates that a drug formulation comprising an effective amount of a β₂-receptor agonist, such as salmeterol or a physiologically acceptable salt thereof, an effective amount of a corticosteroid, such as fluticasone propionate or a solvate thereof, and HFA 134a provides at least one of the following benefits to said patient: less systemic exposure to said drug product and decreased side effects of said drug formulation. This method may comprise the optional additional step of treating a patient with said pharmaceutical formulation.

### BRIEF DESCRIPTION OF THE FIGURES

FIG. 1 shows the median linear plot of plasma fluticasone propionate concentration over time.

FIG. 2 shows a comparative semi-log plot of fluticasone propionate AUCₗₐₛₜ.

FIG. 3 shows a comparative semi-log plot of fluticasone propionate Cₘₐₓ.

FIG. 4 shows the comparative linear plot of fluticasone propionate tₘₐₓ values.

FIG. 5 shows geometric LSMean ratios and associated 90% confidence intervals for Cₘₐₓ and AUC for fluticasone propionate treatment comparison.

FIG. 6 shows the median linear plot of plasma salmeterol concentration over time.

FIG. 7 shows a comparative semi-log plot of salmeterol AUCₗₐₛₜ.

FIG. 8 shows a comparative semi-log plot of salmeterol Cₘₐₓ.

FIG. 9 shows the comparative linear plot of salmeterol tₘₐₓ values.

FIG. 10 shows geometric LSMean ratios and associated 90% confidence intervals for Cₘₐₓ and AUCₗₐₛₜ for salmeterol treatment comparison.

### DETAILED DESCRIPTION OF THE INVENTION

Drugs

Suitable drugs for co-administration by inhalation are also known in the art. Preferred formulations containing combinations of active ingredients contain a β₂-receptor agonist such as salmeterol (e.g., as the xinafoate salt), salbutamol (e.g., as the free base or the sulphate salt) or formoterol (e.g., as the fumarate salt), in combination with an anti-inflammatory steroid such as a fluticasone ester (e.g., the propionate), a beclomethasone ester (e.g., the dipropionate) or budesonide.

A particularly preferred combination is a combination of a topical corticosteroid, such as fluticasone propionate, and a long-acting β₂-receptor antagonist, such as salmeterol,

or a pharmaceutically acceptable salt thereof (particularly the xinafoate salt). A further combination of particular interest is budesonide and formoterol (e.g., as the fumarate salt).

It will be clear to a person skilled in the art that, where appropriate, the drugs may be used in the form of salts, (e.g., as alkali metal or amine salts or as acid addition salts) or as esters (e.g., lower alkyl esters) or as solvates (e.g., hydrates) to optimize the activity and/or stability of the drug and/or to minimize the solubility of the drug in a propellant if desired.

The particle size of the drug in particulate (e.g., micronised) or powder form should be such as to permit inhalation of substantially all of the drug into the lungs upon administration of a aerosol formulation and will thus be less than 100 microns, desirably less than 20 microns, and preferably in the range 1-10 microns, e.g., 1-5 microns.

Propellants

Suitable HFA propellants are known in the art and may be, for example, HFA134a (1,1,1,2-tetrafluoroethane), having the formula CF₃CH₂F, HFA227 (1,1,1,2,3,3,3-heptafluoro-n-propane, having the formula CF₃CHFCF₃, mixtures of HFA134a and HFA227, and the like.

The final inhaler formulation preferably contains 0.005-10% w/w, more preferably 0.005-5.0% w/w, even more preferably 0.01-1.0% w/w, of drug relative to the total weight of the formulation.

Diagnosis, Prescribing Medication, and Treatment

Many patients suffering from asthma attacks generally receive a yearly physical checkup from a general practitioner physician. However, some patients require treatment from an asthma specialist, especially those patients who have severe symptoms and/or receive daily oral corticosteroid treatment.

The medical appointment generally begins with a discussion of the patient's medical history. The physician will ask the patient whether or not the patient has respiratory problems and experiences any of the following physical symptoms: coughing, wheezing, chest tightness, nasal secretions, and allergies. The physician may also ask the patient how long these problems have existed, if they have become progressively worse over time, and if the symptoms are particularly worse at night, which indicates nocturnal asthma. The physician may also ask the patient whether or not the patient's symptoms appear to be linked to an allergen, by asking whether such things as animals, mold, pollen or dust tend to produce asthma attacks. The patient may also be asked to identify other triggers such as stress, exercise, medications, work or home environment, chemicals, smoke, or pollution.

The severity of the asthma can also be determined by finding out if and how often the patient has been hospitalized or treated in an emergency room, or missed work and/or school because of asthma-related illness. The physician will also determine the patient's history of treatment, including whether or not the patient has received prescription medication for controlling asthma.

After the medical history of the patient is assessed, the physician will perform a physical examination in order to definitively diagnose asthma. Some standard procedures used in such as physical examination are: measurement of temperature and pulse, determination of breathing difficulty, listening for breathing difficulty by using a stethoscope, examination of the upper respiratory tract for signs of allergic reactions, such as swelling or tenderness.

The use of machines will also be used to diagnose asthma. The most widely used mechanical test for diagnosing asthma is the lung function test. During this test, the patient breathes into a tube that is attached to a machine. The machine produces a numerical measurement of the patient's forced expiratory volume in one second (FEV₁), which serves to determine the severity of the asthma. Another widely used machine is the peak flow meter, which measures the patient's peak expiratory flow rate (PEFR). This information is especially useful to determining whether or not the patient is responding positively to medication and other treatment.

Finally, the physician will prescribe medication upon taking into account the condition of the patient and knowledge of the possible decreased side effects of medication. The physician may choose to prescribe the inventive inhaler if the patient has a history of a heart condition, such as increased heart rate, sensitive to beta-adrenergic stimulation, and/or cardiac arrhythmia, and whether or not the patient may be or is susceptible to hypercorticism, especially if the physician has been informed of the properties of the composition of the present invention.

Packaged Product

The packaged product of the present invention is made up of a container, such as a box or other suitable packaging, an MDI inside of said container and product information associated with said packaged product. An MDI is a pressurized metered-dose inhaler for oral inhalation, and an exemplary MDI is described in U.S. Pat. No. 6,131,566 (the entire contents of which are incorporated by reference). Packaging for an MDI is described in WO 2000/37336 A1 (the entire contents of which is hereby incorporated by reference). The packaged product can include a flexible package that encompasses the MDI and a desiccant (as described in WO 2000/37336). The suspension of drug in a liquefied propellant such as HFA134a is contained in an aluminum can sealed with a metering valve. The canister is presented to the patient in a plastic actuator fitted with a dust cap.

Product information can be provided in or on the packaging associated with the MDI or on the MDI. Alternatively, the product information can be displayed in close proximity to the MDI. The product information can take the form of an insert (inside the container), a label (on the package or on the MDI), a poster, a compact disk, a floppy disk, or the like. The product information provides a description of the drug inhalation product, including the dosage of drug received in each actuation of the inhaler and the number of actuations provided by the inhaler. The product insert may also provide information describing the clinical pharmacology of the drug, including its mechanism of action, pharmacokinetics, and pharmacodynamics. An indications and usage section of the product insert provides a listing of disease states for which the drug is used as treatment, as well as any contraindications.

A section of the product insert may provide warnings to the patient regarding situations wherein it is not appropriate to use the drug product. For salmeterol, serious acute respiratory events, including fatalities, have been reported when a salmeterol inhalation aerosol has been initiated in a patient with significantly worsening or acutely deteriorating asthma. For fluticasone propionate, particular care is needed for patients who are transferred from systemically active corticosteroids to a fluticasone propionate inhalation aerosol because deaths due to adrenal insufficiency have occurred in asthmatic patients during and after transfer from systemic corticosteroids to less systemically available inhaled corticosteroids.

Adverse reactions may also be described. For salmeterol, adverse reactions are similar in nature to reactions to other selective beta-adrenoceptor agonists, i.e., tachycardia; palpitations; immediate hypersensitivity reaction, including urticaria, angioedema, rash, bronchospasm; headache; tremor; nervousness; and paradoxical bronchospasm. Further, because of the possibility of systemic absorption of inhaled corticosteroids, patients treated with fluticasone propionate must be carefully observed for any evidence of systemic corticosteroid effects, such as hypercorticism (Cushing's disease) and adrenal suppression.

Finally, the product inserts also provide the patient with instructions for use. For maintenance of bronchodilation and prevention of symptoms of asthma, including symptoms of nocturnal asthma, the usual dosage for patients 12 years of age and older is two inhalations twice daily (morning and evening, approximately 12 hours apart). Adverse effects are more likely to occur with higher doses of the drug combination, and more frequent administration or administration of a larger number of inhalations is not recommended.

Suitable daily doses may be, for example, 100 μg of salmeterol and 200 to 2000 μg of fluticasone propionate. Typically, each filled canister for use in a MDI contains 100, 160, or 240 metered doses or puffs of medicament.

Patient Groups

This product may be promoted for use with advertisements, and/or used with various groups of patients who may especially benefit from the product, especially as this product is useful in its ability to lower negative side effects. For example, patients with cardiovascular disease who are sensitive to β-antagonist side effects, patients who are sensitive to inhaled corticosteroids, children under 18 years of age, but old enough to use an MDI, whose growth might be affected by cortisol treatment, or those who require a continuous chronic dose of cortisol, would benefit from the product. Normally, a product insert would explain (or perhaps have data showing) the lessened negative side effects that might be obtained by inhalation of drugs with a HFA propellant, for example, data showing a decreased amount of cortisol in the blood.

This packaged product may be marketed according to methods used in the art. For example, the packaged product may be marketed through the Internet, newspaper, television, or radio advertisements. The packaged product can be shown at trade shows, such as physician conventions.

### EXAMPLES

The below examples are used to exemplify the present invention and are in no way meant to narrow the scope of the invention. The examples compare the systemic pharmacokinetic and pharmacodynamic of a MDI made up of two drugs, namely, salmeterol and fluticasone propionate combined in a HFA propellant, namely 134a, with individual salmeterol and fluticasone propionate MDIs in a CFC propellant administered individually and with placebo (HFA 134a propellant alone). Healthy human subjects were given either salmeterol and fluticasone propionate in HFA 134a propellant, salmeterol in P11/P12, fluticasone propionate in P11/P12, or a placebo in HFA 134a propellant, in a randomized, single dose, crossover study. Potential side effects such as increased heart rate and QTc interval were measured. The levels of cortisol in the urine were also measured as a measure of HPA suppression.

The Examples will now be explained in detail.

Study Groups and Treatment

Twenty healthy human subjects were randomized into one of four treatment groups. Each subject received four single doses according to the random code in a crossover fashion, with seven days in between each dosing session. Subjects received either:

(1) 4 actuations (ex-valve) of salmeterol 25 μg/fluticasone propionate 250 μg combination MDI in HFA 134a propellant (herein referred to as SFC) for a total dose of salmeterol 100 μg/fluticasone propionate 1000 μg, or

(2) 4 actuations of SEREVENT P11/P12 MDI (herein referred to as SALM) containing 25 μg/actuation for a total dose of salmeterol 100 μg, or

(3) 4 actuations of FLOVENT P11/P12 MDI (herein referred to as FP) containing 250 μg/actuation for a total dose of fluticasone propionate 1000 μg, or

(4) a placebo (4 actuations from a placebo MDI containing HFA 134a alone).

Inhalations were given at 30-second intervals over 1.5 minutes. Three strengths (ex-valve) of salmeterol/fluticasone propionate were developed in the HFA 134a MDI: 25 μg/50 μg, 25 μg/125 μg, and 25 μg/250 μg. Of the three strengths, the highest strength (25 μg/250 μg) was used. Corresponding ex-actuator does are: 21 μg/44 μg, 21 μg/110 μg, and 21 μg/220 μg. A 100 μg salmeterol dose and a 1000 μg fluticasone propionate dose were given to provide peak plasma salmeterol levels and a complete plasma fluticasone propionate profile, respectively.

Pharmacokinetic Measures

In order to determine the plasma salmeterol concentrations, four milliliter blood samples were collected pre-dose and for 30 minutes after dosing at 2, 5, 10, 20 and 30 minutes from the beginning of dosing. For the determination of plasma fluticasone propionate concentrations, five milliliter blood samples were collected pre-dose and after dosing at 10 min., 20 min., 30 min., 45 min., 1.0 h., 1.5 h., 2.0 h., 3.0 h., 4.0 h., 6.0 h., 8.0 h., 12 h., 16 h., 20 h., and 24 hours from the beginning of dosing.

Plasma was analyzed for fluticasone propionate and salmeterol concentrations at each time point using solid phase extraction in combination with liquid chromatography tandem mass spectrometry LC-MS-MS. The method has been validated to a limit of quantitation of 20 pg/ml for fluticasone propionate and 0.053 ng/ml for salmeterol.

Pharmacodynamic Measures

Urine was collected for 24 hours pre-dose and for 24 hours post-dose for cortisol determination. Cortisol levels were determined from 500 μl of urine by automated immunochemiluminescence on the ASC-180 (Bayer Diagnostics) following preliminary extraction of the urine with dichloromethane. The method was validated over the range of 6-2069 nmol/l.

Heart rate, systolic and diastolic blood pressure, 12-lead ECG (for QT interval), and 2 ml blood samples for serum potassium and glucose determinations were collected pre-dose and post dose at 5 min., 10 min., 30 min., 1.0 h., 1.5 h., 2.0 h., 3.0 h., and 4.0 hours. Heart rate, blood pressure and 12 lead ECGs were recorded three times before dosing and individual readings were taken at the scheduled times after dosing. Subjects were semi-recumbent, and rested in this position at least 10 minutes before each reading. Pre-dose vital sign measurements were taken every five minutes until three consecutive blood pressure pulse readings were within 10 mmHg and 10 beats per minute, respectively. The mean of the last three consecutive readings was calculated as the baseline value for analysis. Serum potassium and glucose levels were measured using the Synchron CX9 Clinical Analyzer (Beckman).

Pharmacokinetic Analyses

The following parameters were derived for each subject from the plasma fluticasone propionate and salmeterol concentrations by standard non-compartmental analyses using WinNonlin Professions, Version 1.5 (Pharsight Corp., Mountain View, Calif.).

1. Maximum plasma fluticasone propionate and salmeterol concentrations (Cₘₐₓ).

2. Time of Cₘₐₓ(tₘₐₓ).

3. Terminal elimination rate constant for fluticasone propionate (λ_{z}), and the corresponding half-life (t_{1/2}) obtained using concentrations from the log-linear portion of the curve.

4. Area under the plasma fluticasone propionate and salmeterol time curves from zero to the last quantifiable plasma concentration (AUCₗₐₛₜ) calculated using the linear/log trapezoidal method.

5. Area under the plasma fluticasone propionate time curve, extrapolated to infinity time (AUC_{∞}) using the equation (AUCₗₐₛₜ+Cₗₐₛₜ/λ_{z}) where Cₗₐₛₜ is the last measurable plasma concentration.

Actual sampling times were used in the calculation of all pharmacokinetic parameters. Values below the quantitation limit (BQL) of the assay were assigned a value of zero at early time points. When two consecutive BQL values occurred at later time points, all subsequent quantifiable values were excluded from analysis. However, when only one BQL value occurred at a later time point between two measurable concentrations, only the BQL value was excluded from analysis.

The critical endpoints for fluticasone propionate and salmeterol were Cₘₐₓ and AUCₗₐₛₜ. Analysis of AUC, Cₘₐₓ, and t½ was performed after log transformation and tₘₐₓ was analyzed non-parametrically without transformation. Plasma concentration data was listed and summarized by mean, median, standard deviation, minimum and maximum values at each time point for each treatment. Pharmacokinetic parameters were summarized by mean, standard deviation, coefficient variation, median, minimum, maximum value, standard deviation of log transformed data, geodetic mean, and 95% confidence interval for each treatment. Analysis of variance was used to compare between treatments. For comparative purposes, the 90% confidence intervals for the treatment ratios were plotted with the range 0.7-1.43 and used to describe a 30% difference between drug products.

Pharmacodynamic Analyses

The total amount of cortisol excreted was obtained by multiplying the urinary free cortisol concentration by the volume to give the total amount of cortisol excreted over the time period. Concentrations below assay sensitivity (6 nmol/l) were assigned a value of 3 mmol/l. Molar values were converted to micrograms. Both pretreatment and post-treatment values were listed for each subject and were summarized by median, minimum, maximum, mean, standard deviation, coefficient of variation, geometric mean and standard deviation of log transformed data for each treatment. The change and percentage change of post-treatment from pretreatment was listed for each subject and summarized by median, minimum and maximum values for each treatment. Analysis of variance was used to compare between pre and post-treatment allowing for effects due to subject, period and time (pre or post) after log transformation. Analysis of covariance after log transformation including subject, period, treatment as effects and pretreatment measurements as a covariant were also performed for treatment comparisons.

Weighted means for each salmeterol PD parameter (heart rate, systolic and diastolic blood pressure, QTc interval from 12-lead ECG (corrected using Bazett's Formula), serum potassium, and glucose were calculated by dividing the area under the effect-time curve by the sampling interval allowing the parameter to be expressed in units of measure. Area was calculated using the linear trapezoidal method. Maximum of pulse, QTc interval, systolic blood pressure and serum glucose and minimum diastolic blood pressure and serum potassium were also obtained. The mean (geometric mean for serum potassium and serum glucose) was listed for each treatment. Their relationship with treatment group was assessed using analysis of covariance allowing for effects due to subject, period, and treatment and pretreatment measurements as a covariant.

Analysis of variance or covariance using SAS PROC MIXED version 6.12 (SAS Institute Inc., Cary, N.C.) was performed as appropriate including effects due to subject, period, and treatment for all log transformed and untransformed PK and PD parameters as described earlier. For log-transformed parameters the difference in least square means (combination-individual or post-pre) and the 90% (or 95% for PD parameters) confidence interval were back transformed (i.e., exponential transformation) for expression as a ratio (combination as a percentage of the individual). For untransformed parameters, the 90% (or 95%) confidence interval for the difference in least squares means was expressed as a ratio of the individual mean.

Pharmacokinetic Results

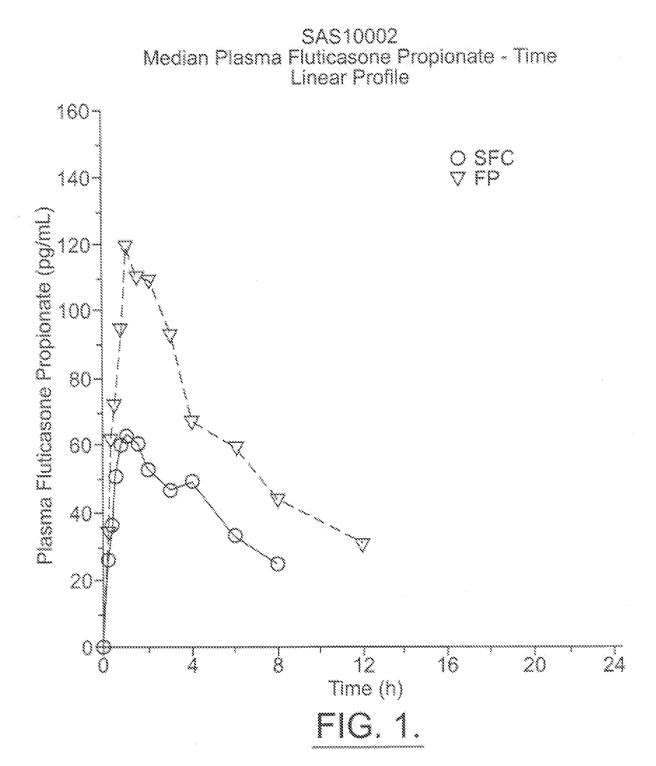

A median linear plot of plasma fluticasone propionate concentrations over time is presented in FIG. 1. As shown in the figure, plasma fluticasone propionate concentrations following SFC administration were consistently lower than after FP administration. The concentrations of both FP and SFC rose sharply within the first hour of treatment with maintained high levels over a period of about 4 hours.

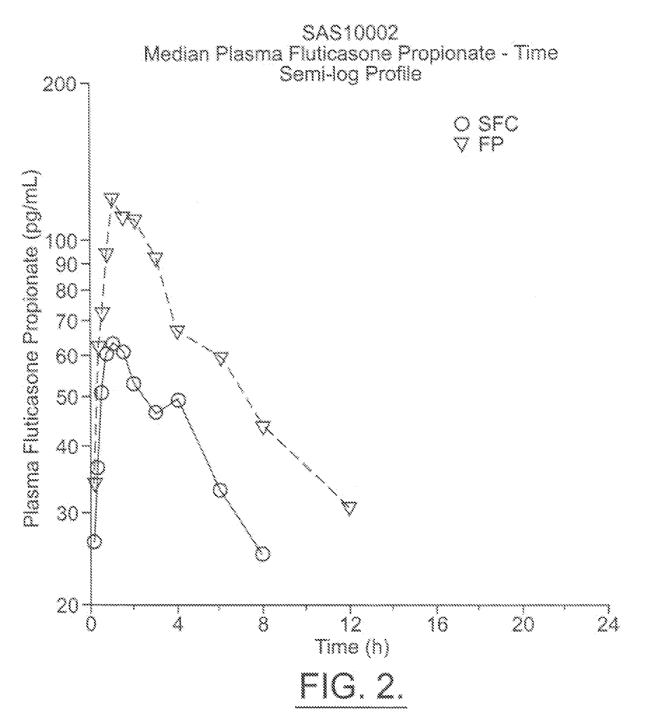

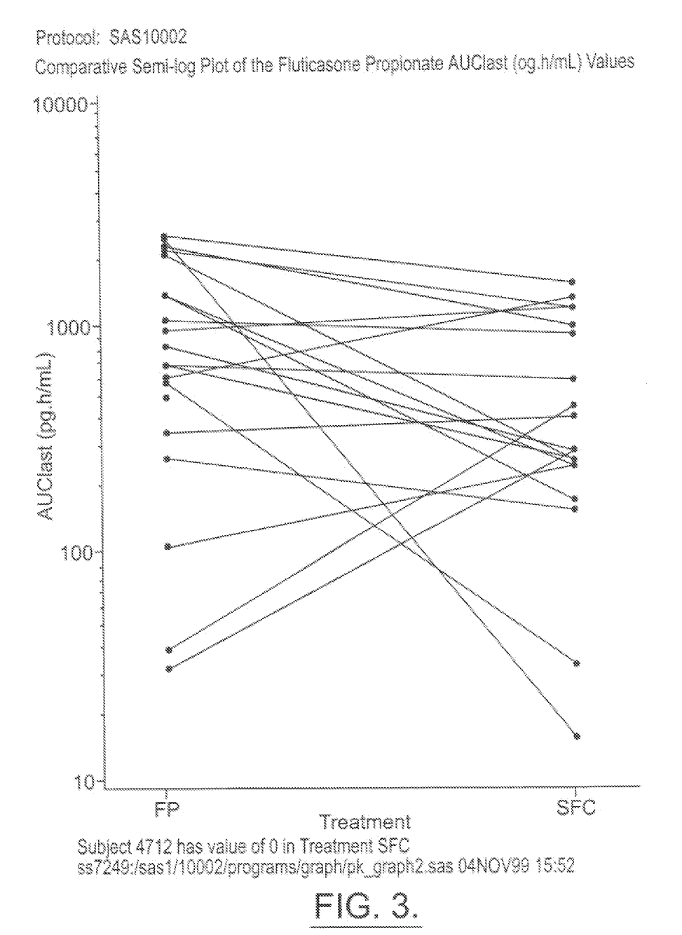

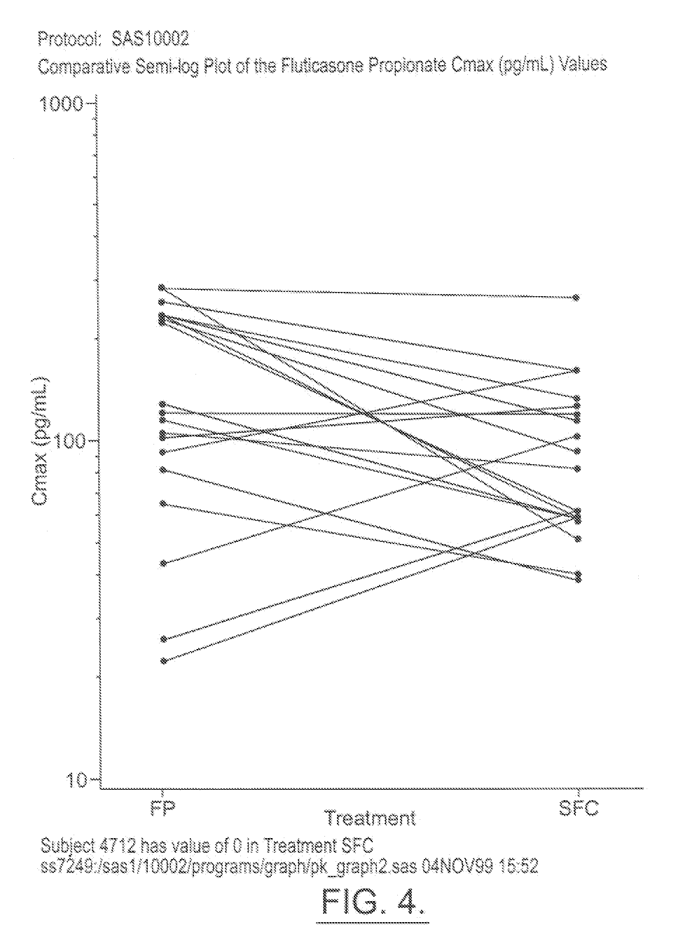

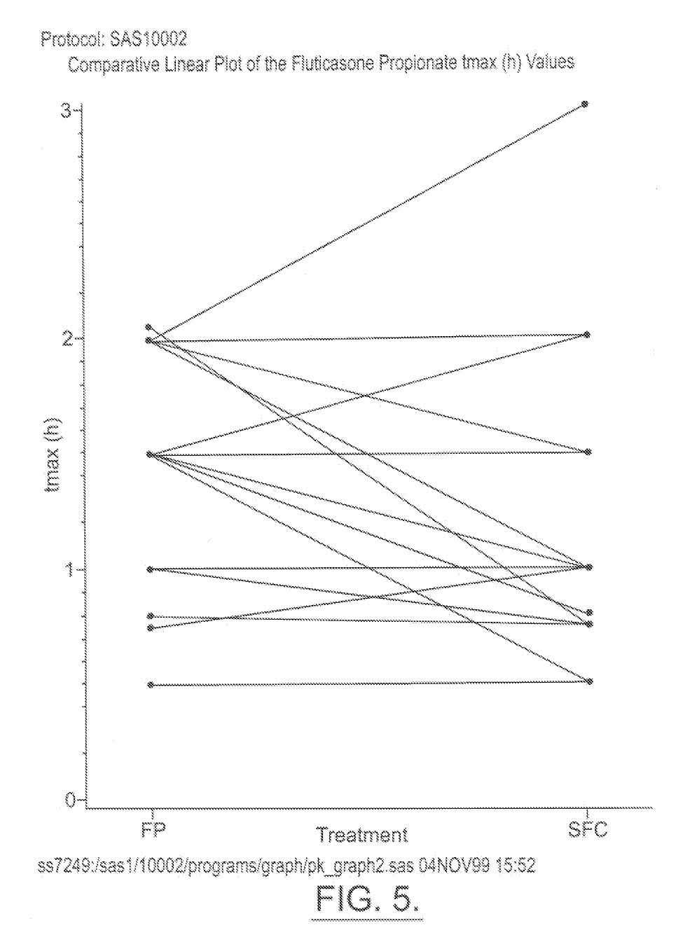

Because concentrations of fluticasone propionate were appreciably lower from SFC, significantly lower AUCₗₐₛₜ and Cₘₐₓ estimates were found when compared to FP. The mean AUCₗₐₛₜ for SFC was 53% of the AUCₗₐₛₜ for FP. Tₘₐₓ, however, was similar following both treatments. Comparative semi-log plots of AUCₗₐₛₜ and Cₘₐₓ from each individual subject (FIGS. 2 and 3, respectively) reflect the lower fluticasone propionate levels following SFC administration observed in most subjects compared to FP administration. FIG. 4 shows the comparative linear plot of fluticasone propionate tₘₐₓ values, showing that tₘₐₓ was similar across treatments. The 90% confidence intervals for the AUCₗₐₛₜ and Cₘₐₓ parameters were considerably outside the range 0.70-1.43 used to describe a 30% difference between treatments, indicating that the pharmacokinetics for the two formulations (SFC and FP) were not comparable for FP (FIG. 5).

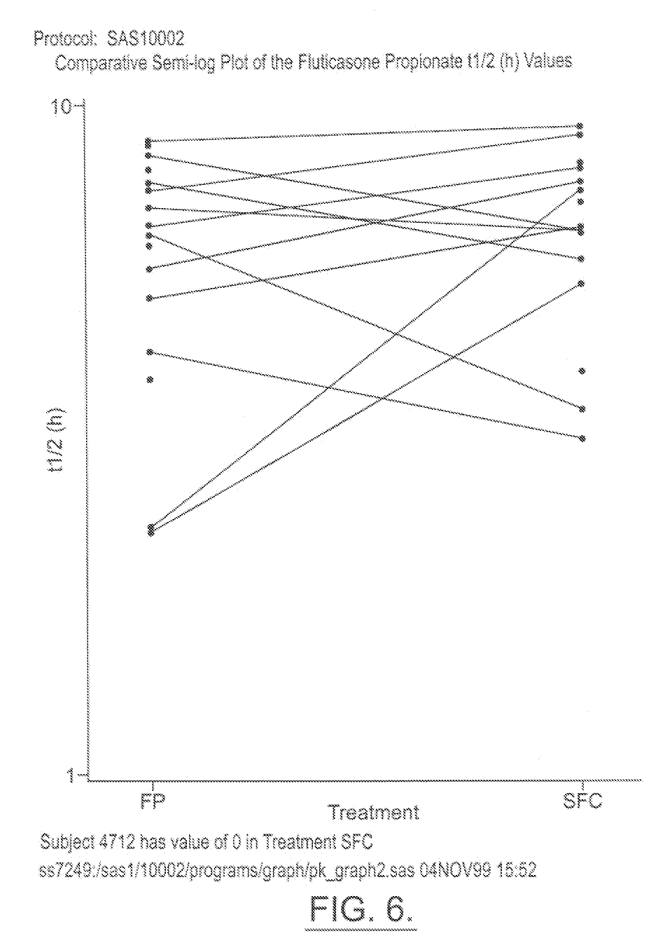

The median linear plot of plasma salmeterol concentrations over time is presented in FIG. 6. As shown in the figure, plasma salmeterol concentrations following SFC administration were consistently lower than after SALM administration. The concentrations of both SALM and SFC rose sharply within a few minutes of dosing, with measurable concentrations usually maintained over the 30-minute sampling period.

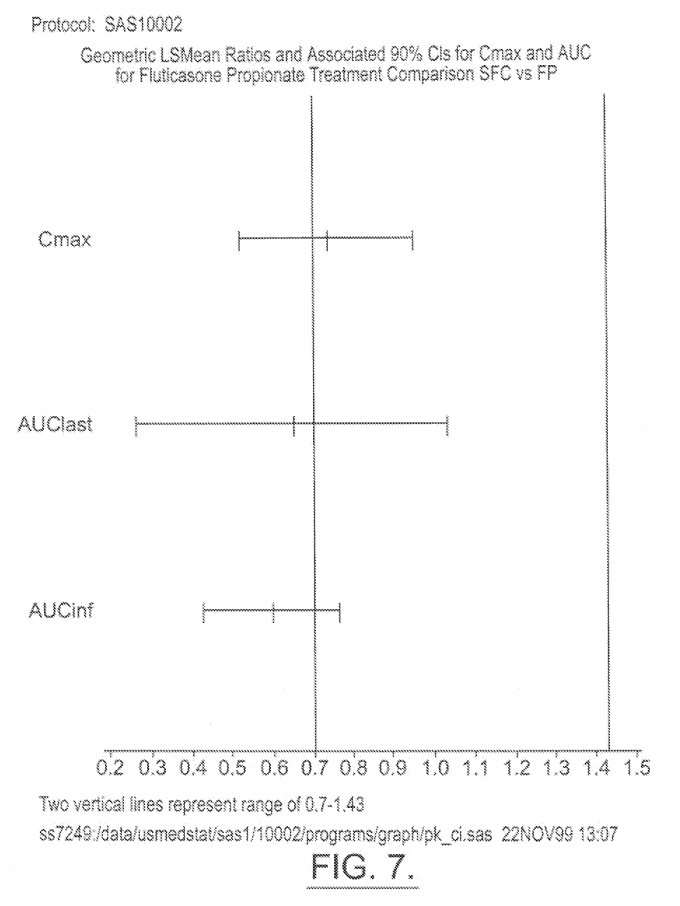

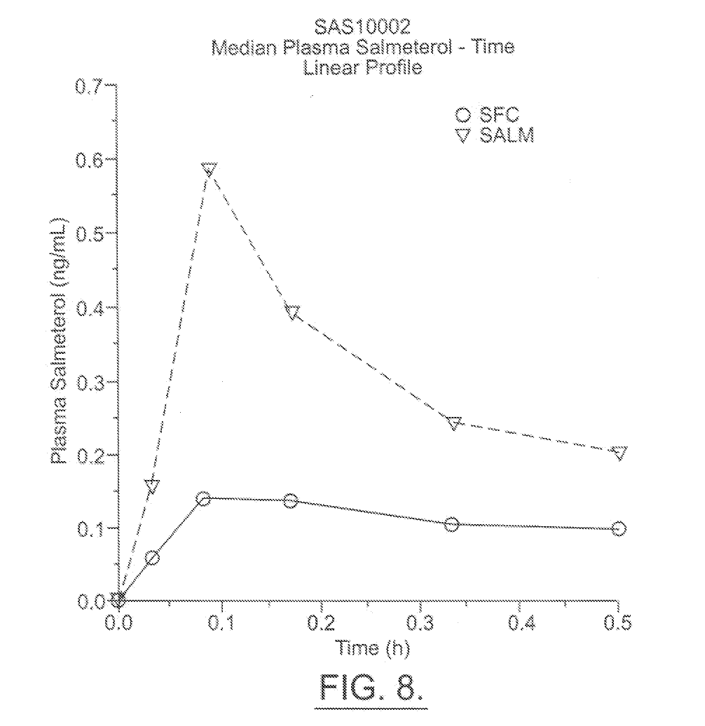

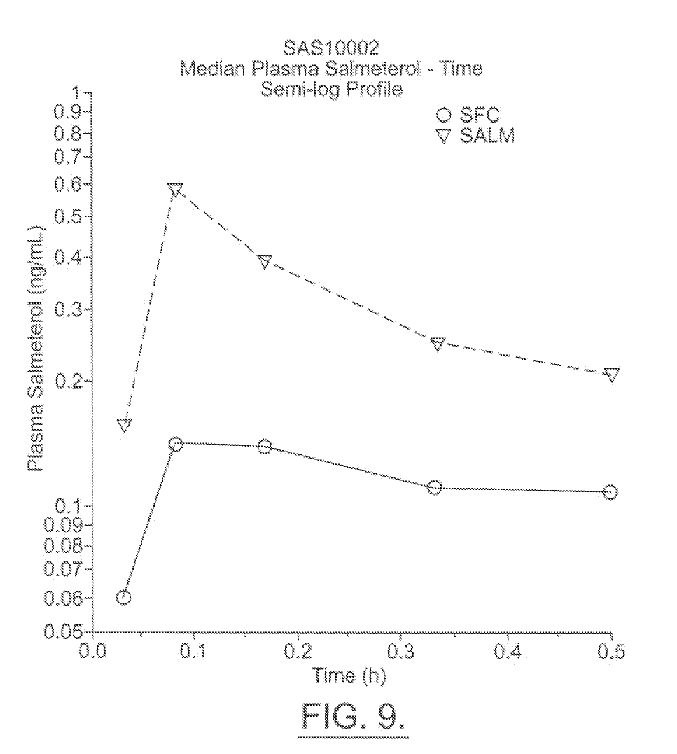

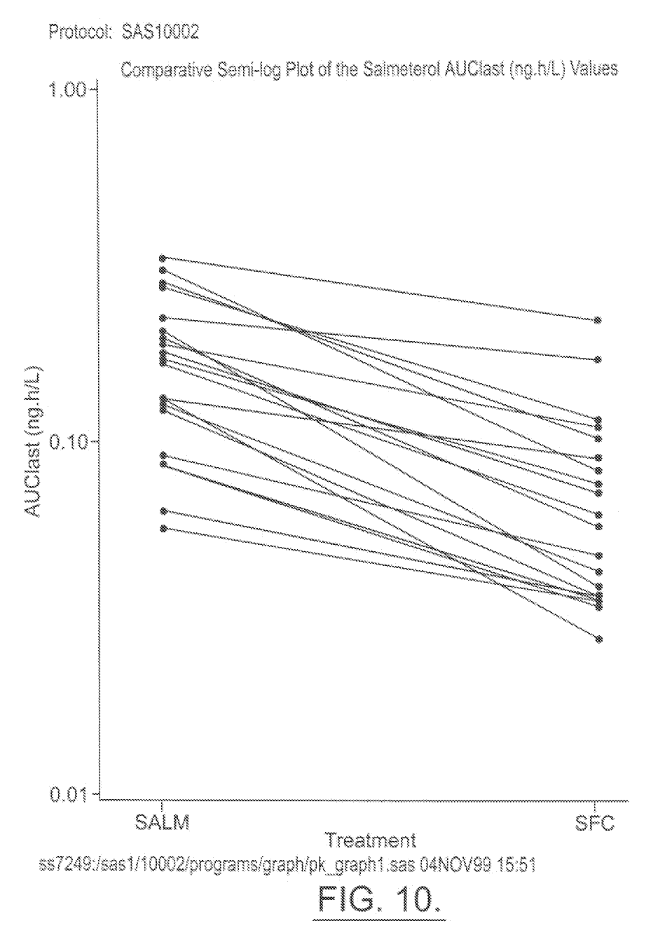

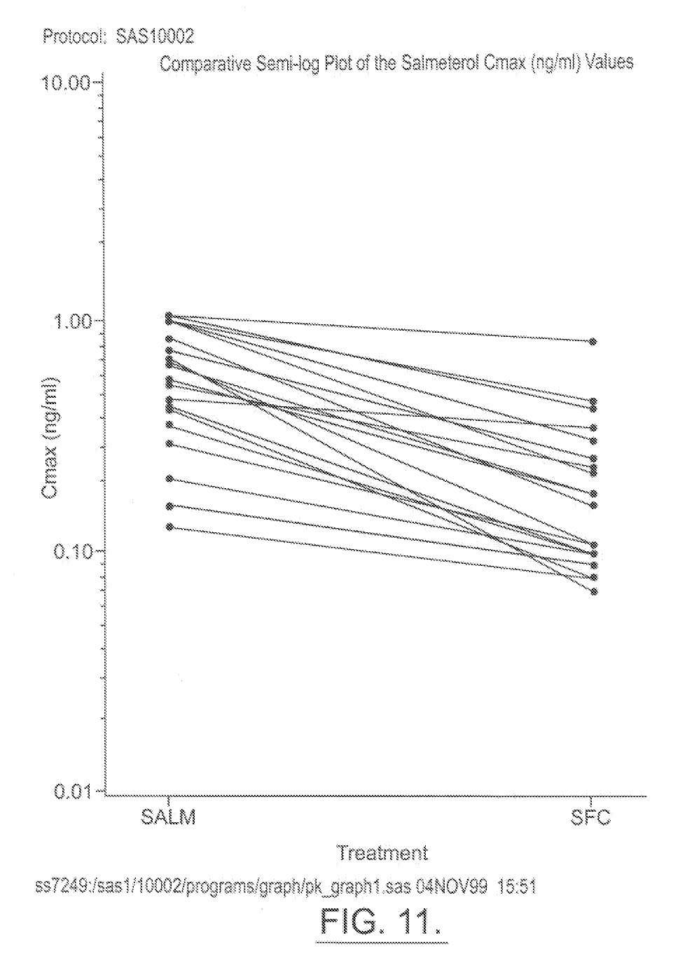

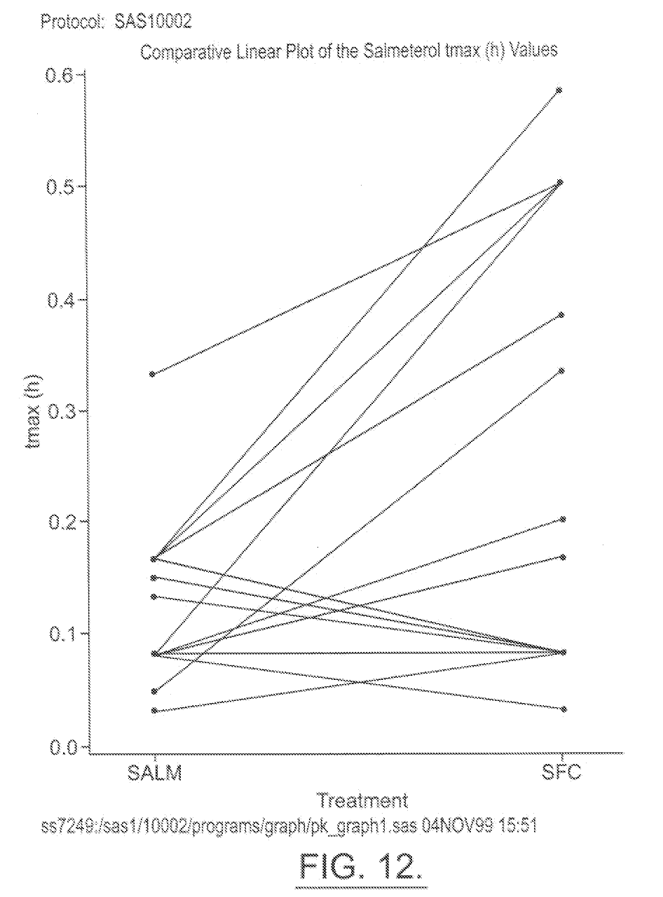

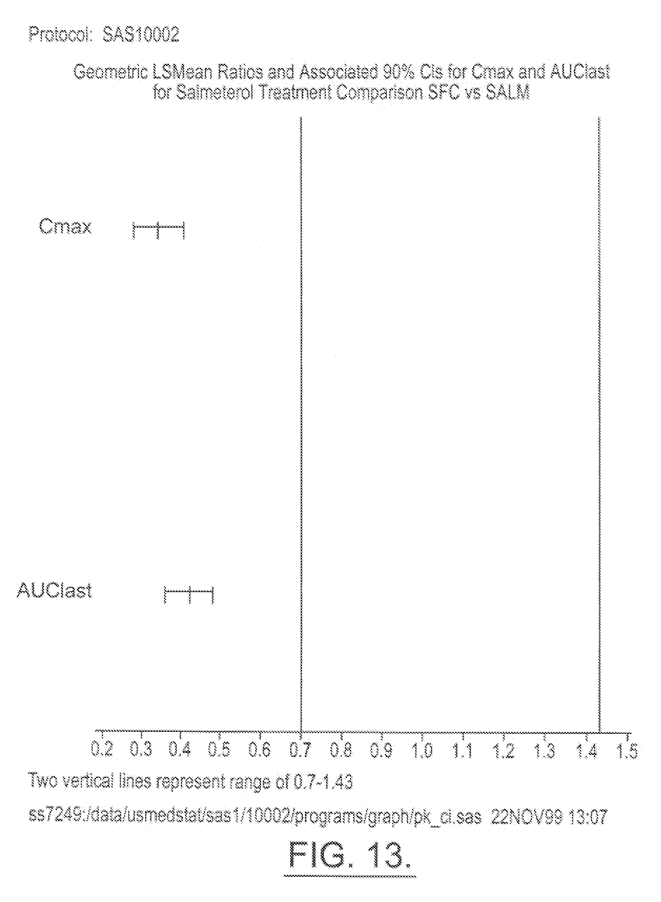

Salmeterol concentrations were appreciably lower from the SFC inhaler resulting in significantly lower AUCₗₐₛₜ and Cₘₐₓ estimates compared to SALM inhaler. Mean AUCₗₐₛₜ for SFC was 42% of the AUCₗₐₛₜ for SALM. Tₘₐₓ was similar. Comparative semi-log plots of individual subject AUCₗₐₛₜ and Cₘₐₓ (FIGS. 7 and 8, respectively) reflect the lower salmeterol levels following SFC administration. FIG. 9 shows the comparative linear plot of salmeterol tₘₐₓ values. The 90% confidence intervals for the AUCₗₐₛₜ and Cₘₐₓ parameters were considerably below the range 0.70-1.43 used to describe a 30% difference between treatments indicating that the pharmacokinetics for the two formulations (SFC and SALM) were not comparable for salmeterol (FIG. 10).

Pharmacodynamic Results

Individual urinary cortisol concentrations and urine volumes over the 24-hour sampling period represent the effect of FP. A significant reduction in cortisol excretion was only observed following FP administration (Table 1). Specifically, urinary cortisol excretion following FP was 64% of placebo. Cortisol excretion was unaffected following SFC or SALM administration when compared to the placebo. Post-treatment geometric means for these treatments ranged between 26.3 to 28.3 μg compared to 18.5 μg for fluticasone propionate resulting in significant differences between FP vs. placebo and FP vs. SFC comparisons (Table 2). In other words, urinary cortisol excretion following SFC and SALM were unchanged from pretreatment levels compared to FP, wherein cortisol excretion was reduced by approximately half.

As discussed below, while blood pressure and serum potassium were unaffected, significant changes in heart rate, QTc, and serum glucose following SFC and SALM compared to placebo were observed.

Mean heart rate over time is shown in FIG. 11. Weighted mean heart rate increased 4.4 to 6.5 beats/min. over placebo following SFC and SALM administration, but not following FP, which only increased 1.1 beats/min. Mean heart rate following SALM (66.1 beats/min) was higher than SFC (64.0 beats/min). Maximum heart rate gave similar results except that the difference between SFC and placebo was not significant.

Mean QTc over time is shown in FIG. 12. Weighted mean QTc for SFC, FP, and SALM increased over placebo. QTc following SALM was higher than after SFC. Maximum QTc for SFC (397.9 msec.) and SALM (401.0 msec.) was higher than placebo (391.3 msec.), but the differences between FP (391.7 msec.) and placebo (391.3 msec.) and between SFC and SALM were not significant.

Weighted mean and maximum serum glucose for SFC (99.9 mg/dl) and SALM (101.4 mg/dl) were similar and higher than placebo (94.6 mg/dl), respectively but not following FP (94.9 mg/dl).

Thus, SFC and SALM produced similar changes in serum glucose and maximum QTc, but SALM produced larger changes in heart rate and weighted QTc, compared to SFC.

In this study, changes in systemic exposure were evaluated by simultaneously evaluating several pharmacodynamic parameters. SFC administration did not affect urinary cortisol excretion as compared to FP administration that produced significant decreases in urinary cortisol. SFC and SALM produced significant changes in heart rate, QTc, and serum glucose, but SFC changes in heart rate and QTc were less than SALM due to lower plasma salmeterol concentrations found after SFC. Thus, SFC in the HFA formulation is less likely to produce these unwanted effects than SALM in the CFC propellant.

Earlier work with the SFC Diskus combination product ruled out a drug-drug interaction and is independent of the inhaler used. Therefore, the lower systemic exposure observed is likely due to biopharmaceutical factors including the different propellants used. The FP and SALM formulations use the CFC propellant P11/12, while SFC utilizes the CFC-free propellant, HFA134a. Thus, it is believed that the co-administration of two drugs with a HFA propellant provided these unexpected results.

Overall, the results of this study show that fluticasone propionate systemic exposure from the salmeterol/fluticasone propionate HFA134a combination product (SFC) was 53% of the systemic exposure of the fluticasone propionate P11/12 MDI (FP). Further, while a significant reduction in urinary cortisol excretion was seen following dosing from a FP inhaler, cortisol excretion following SFC product was unchanged. Concurrently, systemic exposure of salmeterol from the salmeterol/fluticasone propionate HFA134a combination product (SFC) was 42% of the systemic exposure of the salmeterol P11/12 MDI (SALM). This lower systemic exposure resulted in a less effect on heart rate and QTc interval from the SFC product compared to SALM alone.

1. A method for decreasing systemic exposure if a drug combination comprising at least two drugs in a patient, comprising the step of:
administering by inhalation to a patient in need thereof an effective amount of at least two drugs, and HFA propellant.

2. A method for decreasing side effects of a drug combination comprising at least two drugs in a patient, comprising the step of:
administering by inhalation to a patient in need thereof an effective amount of a pharmaceutical composition comprising at least two drugs, and a HFA propellant.

3. The method of claim 1, wherein said at least two drugs are a corticosteroid and a β₂-receptor agonist.

4. The method of claim 1, wherein said at least two drugs are salmeterol or a physiologically active and pharmaceutically acceptable salt thereof, and fluticasone propionate or a physiologically active and pharmaceutically acceptable solvate thereof.

5. The method of claim 1, wherein the HFA propellant is HFA 134a.

6. A method for controlling hypercorticism in a patient, comprising the step of:
administering by inhalation to a patient in need thereof a pharmaceutical composition comprising an effective amount of a β₂-receptor agonist and an effective amount of a corticosteroid, and HFA 134a.

7. The method of claim 6, wherein said β₂-receptor agonist is salmeterol or a physiologically acceptable salt thereof, and said corticosteroid is fluticasone propionate or a solvate thereof.

8. The method of claim 6, wherein the patient is sensitive to hypercorticism.

9. A method for reducing the increase in heart rate in a patient, comprising the step of:
administering by inhalation to a patient in need thereof a pharmaceutical composition comprising an effective amount of a β₂-receptor agonist and an effective amount of a corticosteroid, and HFA 134a.

10. The method of claim 9, wherein said β₂-receptor agonist is salmeterol or a physiologically acceptable salt thereof, and said corticosteroid is fluticasone propionate of a solvate thereof.

11. The method of claim 9, wherein the patient is an asthma patient who may be sensitive to heart rate changes.

12. A method for potentially preventing cardiac arrhythmia or sudden death in a patient, comprising the step of:
administering by inhalation to a patient in need thereof a pharmaceutical composition comprising an effective amount of a β₂-receptor agonist and an effective amount of a corticosteroid, and HFA 134a.

13. The method of claim 12, wherein said β₂-receptor agonist is salmeterol or a physiologically acceptable salt thereof, and said corticosteroid is fluticasone propionate or a solvate thereof.

14. The method of claim 12, wherein the patient has been diagnosed as having a heart condition or sensitive to beta-adrenergic stimulation.

15-18. (canceled)

19. A packaged inhaler for treating asthma, comprising:
an aerosol drug dispensing device:
a pharmaceutical formulation comprising an effective amount of a β₂-receptor agonist and an effective amount of a corticosteroid, and HFA 134a contained in said aerosol drug dispensing device; and
printed information associated with said drug dispensing device which describes at least one of the following:
less systemic exposure to said drug product, and
decreased side effects of said drug formulation.

20. The method of claim 19, wherein said β₂-receptor agonist is salmeterol or a physiologically acceptable salt thereof, and said corticosteroid is fluticasone propionate or a solvate thereof.

21-24. (canceled)
